(19) United States
(12) Patent Application Publication
(10) Pub. No.: US 2025/0276185 A1
(43) Pub. Date: Sep. 4, 2025

(54) SYSTEMS AND METHOD FOR SYNCHRONOUS CIRCUMFERENTIAL INTRAMYOCARDIAL RESYNCHRONIZATION AND PACING

(71) Applicant: THE UNITED STATES OF AMERICA, AS REPRESENTED BY THE SECRETARY, DEPARTMENT OF HEALTH AND HUMAN, Bethesda, MD (US)

(72) Inventors: Robert J. LEDERMAN, Chevy Chase, MD (US); Aravindan KOLANDAIVELU, Potomac, MD (US); Dursun Korel YILDIRIM, Chevy Chase, MD (US); Christopher G. BRUCE, Washington, DC (US)

(21) Appl. No.: 18/863,596

(22) PCT Filed: May 5, 2023
(86) PCT No.: PCT/US2023/021238

§ 371 Date: Nov. 6, 2024

Related U.S. Application Data
(60) Provisional application No. 63/364,319, filed on May 6, 2022.

(51) Int. Cl. A61N 1/368; A61N 1/05

(52) U.S. Cl. CPC A61N 1/36842; A61N 1/056

(57) ABSTRACT

Various systems and methods are provided for cardiac resynchronization therapy. In one example, apparatus for a multi-electrode pacemaker lead, comprises a plurality of ring electrodes distributed along a length of the multi-electrode pacemaker lead, the multi-electrode pacemaker lead configured to be implanted within myocardium of a wall of a ventricle of a heart such that an outer surface defining a circumference of at least one of the plurality of ring electrodes is in direct contact with the myocardium.

### CROSS REFERENCE TO RELATED APPLICATIONS

The present application is U.S. National Phase of International Application No. PCT/US2023/021238, entitled “SYSTEMS AND METHOD FOR SYNCHRONOUS CIRCUMFERENTIAL INTRAMYOCARDIAL RESYNCHRONIZATION AND PACING,” and filed on May 5, 2023. International Application No. PCT/US2023/021238 claims priority to U.S. Provisional Application No. 63/364,319, entitled “SYSTEMS AND METHOD FOR SYNCHRONOUS CIRCUMFERENTIAL INTRAMYOCARDIAL RESYNCHRONIZATION AND PACING,” and filed May 6, 2022. The entire contents of each of the above-referenced applications are hereby incorporated by reference for all purposes.

### FIELD

The present description relates generally to medical devices for cardiac resynchronization, and method of operation and implantation of the medical devices.

### BACKGROUND/SUMMARY

The contraction and relaxation of cardiac muscle, also called myocardium, results from muscle depolarization and repolarization, which is triggered by electrical impulses. Cardiac resynchronization therapy (CRT) aims to mitigate dyssynchronous cardiac contraction that may be caused by an intrinsic conduction system disease, a myocardial scar, or cardiac pacing therapy. Conventional CRT includes coordinated pacing of the right and left ventricles. CRT left ventricular pacing sites are constrained by cardiac venous anatomy, which may not correspond with optimal pacing sites. Pacing sites may be suboptimal because of abnormal, nonconductive, or scarred myocardium in the vicinity of the pacing site in addition to poor or unreliable electrode-myocardium contact. Moreover, CRT relies on a small number of simultaneous pacing sites. These factors contribute to suboptimal clinical response rates as low as 60-70%.

Similarly, conventional single-site right-ventricular (RV) pacing induces non-physiological cardiac contraction patterns that induce cardiomyopathy. Recent alternatives to single-site RV pacing include direct or nearby pacing to the His bundle or to the left bundle branch (LBB) of the innate cardiac conduction system. However, His bundle pacing is associated with high pacing thresholds and both are subject to lead dislocation. Moreover, His bundle and LBB pacing utilize an intact cardiac conduction system, so these approaches may not be viable for individuals with an impaired or abnormal cardiac conduction system. Furthermore, conventional pacing lead implantation approaches may risk chronic injury to the tricuspid valve.

In one example, the issues described above may be addressed by an apparatus for a multi-electrode pacemaker lead comprising a plurality of ring electrodes distributed along a length of the multi-electrode pacemaker lead, the multi-electrode pacemaker lead configured to be implanted within myocardium of a wall of a ventricle of a heart such that an outer surface defining a circumference of at least one of the plurality of ring electrodes is in direct contact with the myocardium. In this way, contact variability between the myocardium and the ring electrodes may be reduced while a risk of electrode dislodgment is also decreased. In addition, the circumferential distribution of multiple electrodes simultaneously activates the myocardium to reduce dyssynchrony.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 depicts an embodiment of a pacing system comprising a multi-electrode lead that encircles the left ventricle of a heart;

FIGS. 2A and 2B show schematic cross-sectional views of a multi-electrode lead implanted in the wall of the left ventricle of a heart;

FIG. 3 schematically shows synchronous multi-site pacing from a multi-electrode lead implanted in the wall of the left ventricle of a heart;

FIG. 4 schematically shows placement of a multi-electrode lead within the intraventricular septum proximate to the cardiac conduction system;

FIG. 5 schematically shows a first example of bipolar pacing pairs of a multi-electrode lead;

FIG. 6 schematically shows a second example of bipolar pacing pairs of a multi-electrode lead;

FIG. 7 depicts exemplary dimensions for an embodiment of a multi-electrode lead that may be implanted in the wall of the left ventricle of a heart;

FIG. 8 depicts exemplary dimensions for another embodiment of a multi-electrode lead that may be implanted in the wall of the left ventricle of a heart;

FIG. 9 shows a flow-chart of an exemplary method for implanting a multi-electrode lead within myocardium;

FIG. 10 shows a flow-chart of an exemplary method for optimizing an intramyocardial multi-electrode circumferential pacing system; and

FIG. 11 shows a flow-chart of an exemplary method for operating a pacing system comprising a multi-electrode lead that encircles the left ventricle of a heart to provide cardiac resynchronization therapy.

### DETAILED DESCRIPTION

The following description relates to systems and methods for cardiac resynchronization therapy (CRT). In a normal heart, contraction of the left ventricle is coordinated by interaction of intrinsic cardiac conduction tissue and healthy myocardium so that the contraction occurs synchronously from base-to-apex throughout a given layer of the myocardial wall and by a similar amount in order to effectively pump blood. This contraction pattern may be disrupted via dysfunction of the cardiac conduction system (e.g., left bundle branch block), due to scarring in the myocardium, or due to electrical stimulation at a single site produced via cardiac pacing therapy. As a result, cardiac dyssynchrony may occur, wherein portions of the left ventricle may contract at different timings. Cardiac dyssynchrony may be identified, for example, based on incoordinate segmental left ventricle wall motion on imaging studies (such as echo, cardiac magnetic resonance imaging, etc.), and, for example, by incoordinate left ventricular electrical activation causing a wide QRS complex in electrocardiograms. CRT aims to mitigate dyssynchronous cardiac contraction by providing electrical impulses to stimulate coordinated contraction of the left ventricle.

However, conventional pacemaker leads for CRT, and defibrillatory leads for implanted defibrillators, suffer unpredictable and variable tissue-electrode contact zones, which can cause high or variable sensing and stimulation thresholds. In addition, conventional pacemaker leads may produce non-target pacing, such as stimulation of the phrenic nerve. Furthermore, the conventional pacemaker leads may include variable anchoring based on an arbitrary “landing zone” for the lead requiring implantation excessively far from the ventricular base. In addition, bipolar pacing configurations may rely on one intra-vascular and one myocardial electrode, which may increase current requirements for myocardial stimulation. As another example, conventional bundle of His (also referred to herein as a “His bundle”) and left bundle branch (LBB) pacing systems may have insufficient intramyocardial lead positioning trajectories and therefore undergo repeated insertions and removals for site selection during implantation. With imperfect positioning, pacing output may be increased to capture conduction tissue with deleterious consequences on generator battery life. Additionally, there is a higher risk of injury to or interference with tricuspid valve function. Furthermore, the conventional bundle of His and LBB pacing systems may suffer unstable implantation positions that increase an occurrence of lead dislodgement or perforation.

Thus, according to embodiments described herein, synchronous circumferential pacing (SCIRP) may be employed via a multi-electrode lead that is embedded within the myocardium. By utilizing intramyocardial positioning, ring electrodes of the multi-electrode lead have increased contact with the myocardial wall for reduced contact variability. They may also have a reduced risk of infection, although most such infections involve the generator pocket and the intravascular portion of the pacemaker lead. Furthermore, the intramyocardial deployment avoids non-target depolarization, such as undesirable phrenic nerve stimulation. As another example, a plurality of ring electrodes may be implanted in array fashion around the myocardium, enabling both multiple unipolar and multiple bipolar configurations that may be used to overcome zones of poor excitation or high pacing thresholds. It should be understood that the term “plurality” denotes more than one. Further still, the multi-electrode lead may be implanted in proximity to the cardiac conduction system within the interventricular septum to accomplish His bundle or LBB pacing with a reduced risk of dislodgement and without interfering with tricuspid valve leaflet integrity and excursion. Overall, positive patient outcomes may be increased.

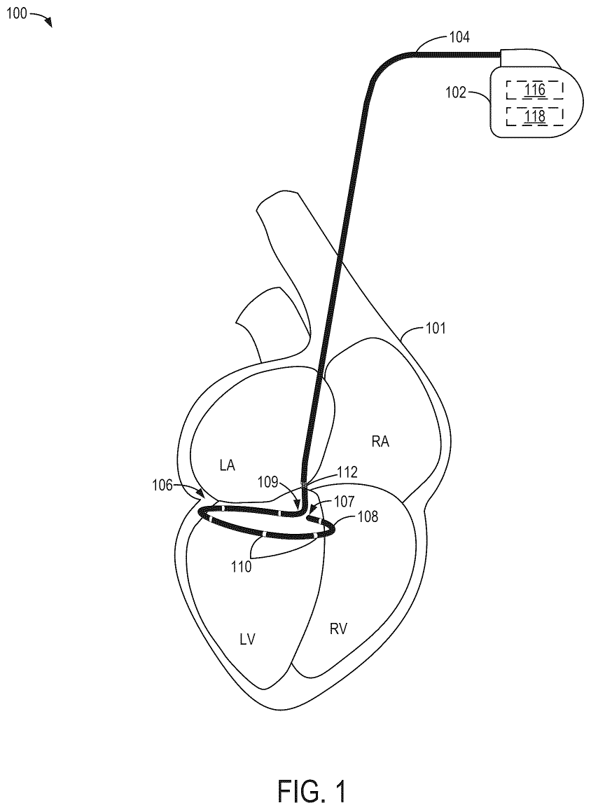

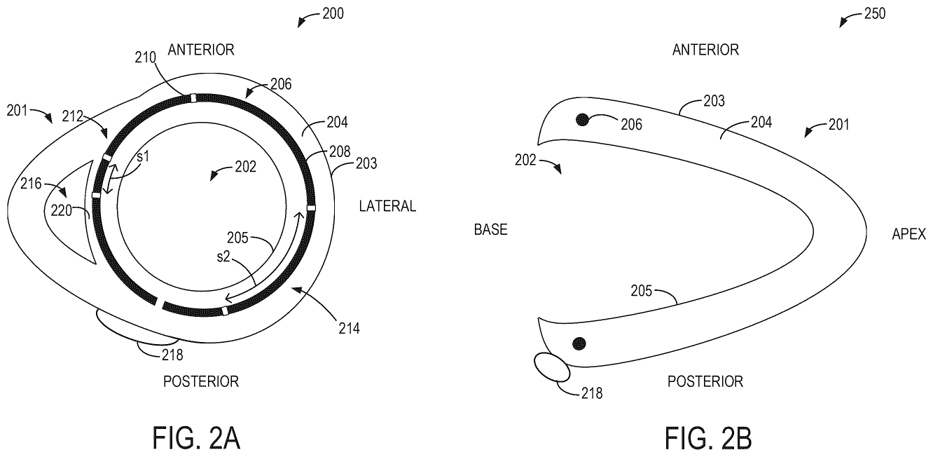

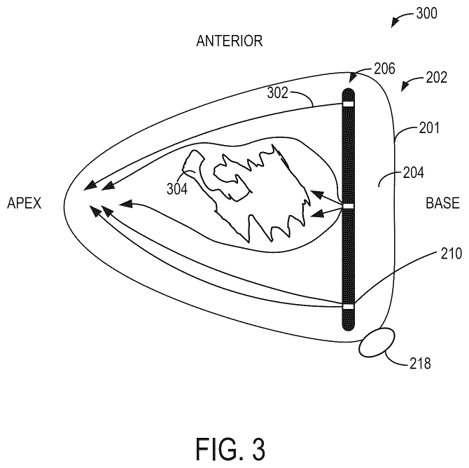

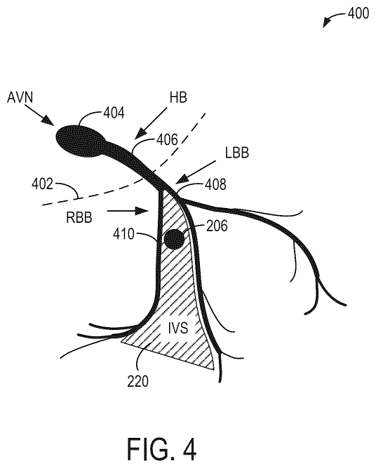

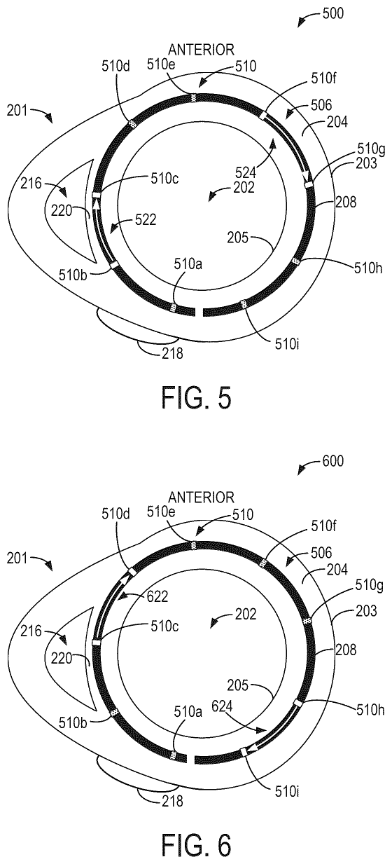

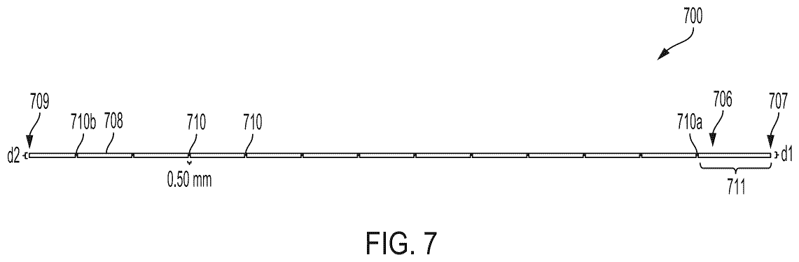

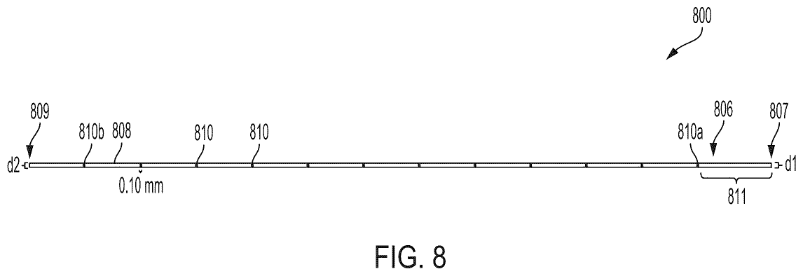

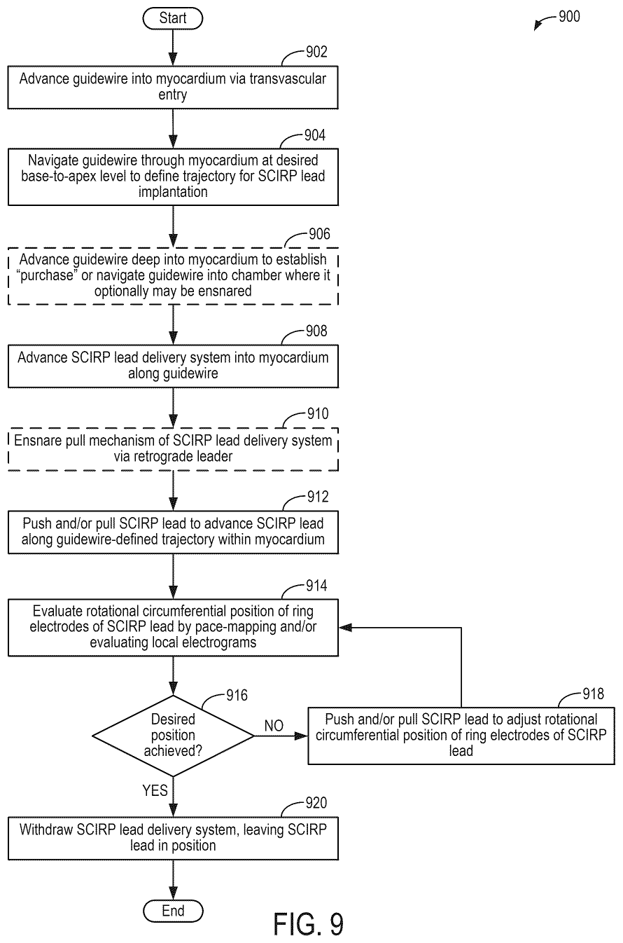

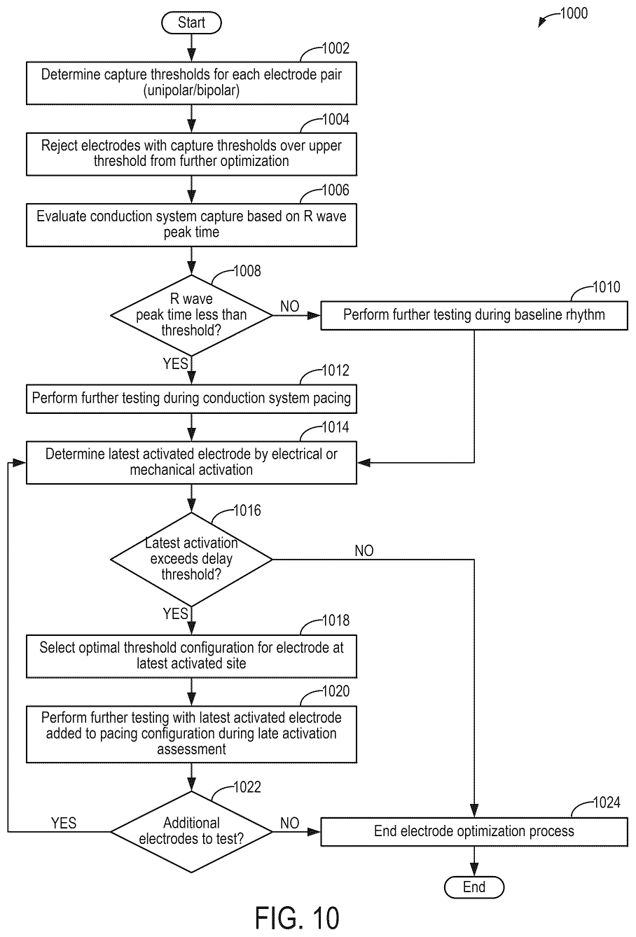

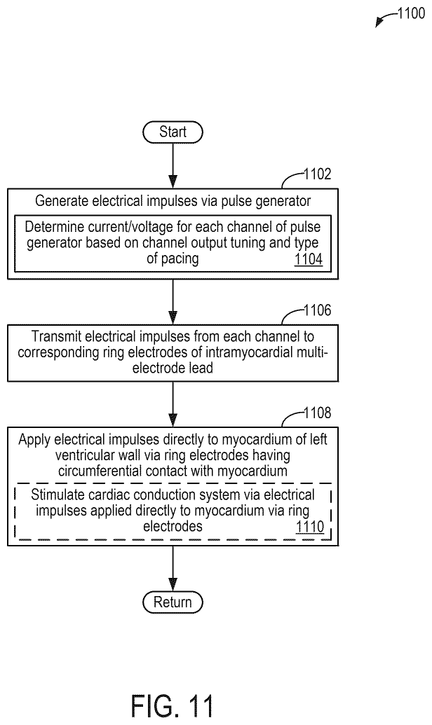

FIG. 1 shows an embodiment of a pacing system comprising a multi-electrode lead embedded within the myocardial wall of the left ventricle of a heart. FIGS. 2A and 2B show schematic cross-sectional views that further depict the intramyocardial positioning of the multi-electrode lead as well as how an electrode density may be varied across the lead body in order to provide desired pacing effects. For example, a higher electrode concentration near or within the ventricular septal conduction system, diagrammatically shown in FIG. 4, may enable His bundle or LBB pacing with reduced placement difficulty. The multi-electrode lead provides an array of ring electrodes around the myocardium to stimulate base-to-apex pacing throughout the left ventricle even in the presence of non-conductive scar tissue, such as schematically shown in FIG. 3. Further, the multi-electrode lead may be adapted to a plurality of pacing configurations, including the exemplary bipolar pacing configurations shown in FIGS. 5 and 6. FIGS. 7 and 8 show exemplary dimensions of the multi-electrode lead, which includes ultra-narrow (e.g., less than 1 millimeter) ring electrodes distributed along the length of the lead body as well as features that may aid implantation. FIGS. 7 and 8 are shown approximately to scale. The multi-electrode lead may be implanted in myocardium around the left ventricle via the method of FIG. 9, for example, and optimized via the method of FIG. 10. Once implanted, the multi-electrode lead may provide synchronous pacing around the circumference of the myocardium for CRT according to the method of FIG. 11.

Turning now to the figures, FIG. 1 schematically depicts an example of a circumferential pacing system 100 with respect to a heart 101 of a patient. The heart 101 is shown as a simplified cartoon including the relative positioning of the left ventricle (LV), right ventricle (RV), left atrium (LA) and right atrium (RA). The circumferential pacing system 100 includes a pulse generator 102 that is coupled to a multi-electrode lead 106. A body 108 of the multi-electrode lead 106 is shown implanted entirely within a wall of the heart 101 and encircling the left ventricle. As described further below in regard to FIGS. 2A and 2B, entry and exit sites where the electrode lead 106 respectively enters and exits the myocardium may vary. As such, the pathway of the electrode lead 106 into the heart 101 may be different than shown schematically in FIG. 1. As an example, the electrode lead 106 may extend through veins to the right atrium. From the right atrium, the electrode lead 106 may extend to the left ventricle via the coronary sinus (e.g., coronary sinus 218 of FIGS. 2A and 2B). Alternatively, from the right atrium, the electrode lead 106 may extend to the left ventricle via the interventricular septum. Alternatively, from the right atrium, the electrode lead 106 may extend to the left ventricle via the right ventricle and the interventricular septum. Other pathways of the electrode lead 106 are also possible, according to the entry and exit sites. As will be further described below, the body 108 refers to a portion of the multi-electrode lead 106 that is implanted in the myocardium and includes a plurality of ring electrodes 110, while a connecting portion 104 of the multi-electrode lead 106 connects the body 108 to the pulse generator 102. In particular, the body 108 is implanted within myocardium surrounding the left ventricle of the heart 101, as will be elaborated below with respect to FIGS. 2A and 2B, and the connecting portion 104 includes insulated conductors (e.g., wires) that electrically couple the plurality of ring electrodes 110 to the pulse generator 102. Further, the pulse generator 102 is an implantable device such that the pulse generator 102 and the multi-electrode lead 106 may be positioned entirely within the patient's body, completely within the heart, or may be connected wirelessly to remotely-positioned generator systems.

The body 108 of the multi-electrode lead 106 is configured to follow the curvature of the left ventricle of the heart 101 when implanted and may substantially encircle the left ventricle along a short-axis trajectory of the heart. In the example shown in FIG. 1, the body 108 surrounds the entire circumference of the left ventricle, with a first end 107 positioned immediately adjacent to a second end 109. However, in other examples, the body 108 may be sized to surround a portion of the left ventricle, with the first end 107 spaced apart from the second end 109 by a desired amount.

For example, the body 108 may be sized to surround approximately 180-360 degrees (e.g., 270 degrees or more) of the circumference of the left ventricle. Unlike pacing systems used in conventional CRT, which may utilize leads terminating in electrodes extending to both the left ventricle and the right ventricle, the circumferential pacing system 100 provides multi-site synchronous pacing to the wall of the left ventricle and the interventricular septum (IVS), which separates the left ventricle and the right ventricle. Due to the synchronous multi-site pacing around the circumference of the left ventricle, the multi-electrode lead 106 may also be referred to as a synchronous circumferential pacing (SCIRP) lead herein.

While the examples illustrated herein show an approximately circular multi-electrode lead trajectory around the ventricle, the path of the multi-electrode lead in traversing at least partly around the ventricle may be sinuous, including straight and non-straight portions, and/or combinations thereof. For example, the path of the multi-electrode lead in traversing at least partly around the ventricle may be comprised of a plurality of segments connected by a plurality of bends. In another example, the path of the multi-electrode lead in traversing at least partly around the ventricle may be comprised of a plurality of curved segments, each with potentially different radiuses of curvature. In still other examples, the path of the multi-electrode lead in traversing at least partly around the ventricle may be maintained on a single flat plane, while yet other examples may include a three-dimensional path that cannot be maintained in a single flat plane.

The multi-electrode lead 106 comprises the plurality of ring electrodes 110, which are distributed along the body 108. For example, the multi-electrode lead 106 may comprise six ring electrodes 110, such as in the example shown. In other examples, the multi-electrode lead 106 may comprise more or fewer than six ring electrodes, such as a number of ring electrodes in a range from 1-16. As one example, the multi-electrode lead 106 may include at least two ring electrodes 110. As another example, the multi-electrode lead 106 may include at least four ring electrodes 110. The ring electrodes 110 may be gold coated, stainless steel, nickel-titanium alloy (e.g., Nitinol), nickel-cobalt alloy (e.g., MP35N), cobalt superalloy (e.g., 35N LT), silver, platinum and platinum-iridium, and/or carbon conductive rings, for example, and each ring electrode 110 provides a terminal of the circumferential pacing system 100 that is in contact with the myocardium.

Each of the plurality of ring electrodes 110 has a cylindrical shape where the circumference (e.g., outer perimeter surface) of the cylinder is in contact with, and completely surrounded by, the myocardium of the heart 101 when the body 108 is implanted. Each ring electrode 110 has a length in a dimension parallel to a central axis of the cylindrical shape. The length of each ring electrode 110 in the multi-electrode lead 106 may be uniform, or the ring electrodes 110 may have varying length. The length of each ring electrode 110 may be less than 1 millimeter (mm). For example, the length may be 0.5 mm or less. As another example, the length may be in a range from 0.1 to 0.2 mm. As still another example, the length of at least one of the ring electrodes 110 may be 0.1 mm or less. All of plurality of ring electrodes 110 of the multi-electrode lead 106 may have the same length. Alternatively, the length of the ring electrodes 110 may be variable such that different ring electrodes have different lengths, as will be elaborated below. Further, in some embodiments, edges of each of the ring electrodes 110 (e.g., a first edge of the length and a second edge of the length) may be coated with a polymer. For example, charge may otherwise concentrate at the edges, and the polymer coating may mitigate this charge accumulation.

The ring electrodes 110 are distributed along the body 108 at a fixed or variable spacing from the first end 107 to the second end 109 around the circumference of the left ventricle of the heart 101, which may be 10-30 centimeters. For example, each of the ring electrodes 110 may be equidistant from neighboring ring electrodes 110 along the multi-electrode lead 106. Alternatively, a distance between neighboring ring electrodes 110 may vary based on a desired spacing of the ring electrodes within the heart 101 when implanted, such as will be elaborated below with respect to FIG. 2A. In some embodiments, the multi-electrode lead 106 may further include a tip electrode that has a surface area comparable to any of the plurality of ring electrodes 110. In some embodiments, the variable inter-electrode spacing may be configured to generate intramyocardial unipolar pacing at one or multiple of the plurality of ring electrodes 110 or bipolar pacing between at least one cathode of the plurality of ring electrodes 110 and at least one anode of the plurality of ring electrodes 110.

In the bipolar pacing configuration, one or more of the ring electrodes 110 may be configured as a common intramyocardial return electrode (e.g., anode) for bipole-at-a-distance pairings with one or more of the other ring electrodes 110. The common intramyocardial return electrode may have a greater cylinder length than the other ring electrodes 110 (e.g., cathodes). The common intramyocardial return electrode may also have a greater surface area compared with the cathode ring electrodes 110 to reduce bipolar power usage and/or reduce anodal capture by reducing the return path resistance. When there is a single larger bipolar return electrode, it may be positioned in the septum near the His Bundle or left bundle to optimize and enhance its performance. The larger length bipolar return electrode may be the distal electrode on the body 108, for example. Alternatively, an additional or second larger length bipolar return electrode may be positioned in the posterior base.

As one example, the distance between each of the plurality of cathode ring electrodes 110 from the common intramyocardial return electrode may range from 15 millimeters to 200 millimeters, which may provide power saving while still providing sufficient contact area for delivery of the electrical stimulation. As another example, a maximum spacing distance between each of the plurality of cathode ring electrodes 110 from the common intramyocardial return electrode may be equal to a minimum spacing distance multiplied by one less than a number of ring electrodes included in the plurality of ring electrodes 110. As still another example, the minimum spacing distance may be in a range between 5 and 20 millimeters (e.g., approximately 14-15 millimeters). For example, the minimum spacing distance may be calibrated to provide effective His bundle or left bundle branch stimulation via an array of ultra-narrow ring electrodes 110 rather than one larger electrode, as the ultra-narrow ring electrodes 110 may have reduced power consumption compared with the one larger electrode. Example dimensions of the ring electrodes 110 will be described below with respect to FIGS. 7 and 8.

The plurality of ring electrodes 110 may include a surface treatment, such as a fractalized surface treatment. The fractalized surface treatment may decrease myocardial stimulation thresholds and prolong pulse generator battery life (e.g., the battery life of the power source 116). For example, including a fractal coating may enhance electrical properties of the plurality of ring electrodes 110, such as by reducing polarization losses associated with the capacitance of small surface area electrodes. Further, the fractalized surface treatment may increase an electrochemical surface area of each of the ring electrodes 110. Additionally or alternatively, the surface treatment may comprise a drug-eluting ring surface, such as a steroid-eluting ring surface (e.g., a glucocorticoid-eluting surface). The use of steroid elution may help suppress myocardial inflammation to lower pacing thresholds and mitigate variable pacing and sensing thresholds, for example.

The body 108 of the multi-electrode lead 106 comprises one or more tube-shaped materials having a desired stiffness (or flexibility) and compressive strength, enabling the multi-electrode lead 106 to flex with the mechanical movement of the heart 101 while maintaining its shape between heartbeats. For example, the tube-shaped material may be a bioinert or biocompatible polymer, thermoplastic polymer, polyester, plastic, elastomer, or a combination of such materials. As one example, an outer surface of the body 108 may comprise Pebax® tubing. As another example, the outer surface of the body 108 may comprise polyether-ether ketone (PEEK) tubing. An inner surface (e.g., inner lumen) of the body 108 may comprise a thin polymeric lining, such as a thin-wall polyamide tube or a polytetrafluoroethylene (PTFE) liner. Relative dimensions of the body 108 will be described below with respect to FIGS. 7 and 8.

The body 108 may provide a substrate for the plurality of ring electrodes 110 and also house transmission lines (e.g., wires) for each of the ring electrodes 110. For example, each of the ring electrodes 110 may be connected to the pulse generator 102 via individual enamel-coated copper wires, and the body 108 may provide further insulation for the copper wire conductors. As such, the plurality of ring electrodes 110 may undergo individual impedance-matching of each electrode channel at the pulse generator 102. The enamel-coated copper wires may be 38 gauge (according to American Wire Gauge system), for example. The enamel-coated copper wire connected to each of the ring electrodes 110 may be encased by the body 108 and extend through at least a portion of the body 108 before exiting the body 108 at a wire outlet at the second end 109. The wire outlet may direct the enamel-coated copper wires into the connecting portion 104 of the multi-electrode lead 106 to electrically couple (e.g., connect) the plurality of ring electrodes 110 to the pulse generator 102. The connecting portion 104 may be comprised of the same or a similar material as the body 108 of the multi-electrode lead 106 (e.g., a tube-shaped bioinert thermoplastic polymer or elastomer). Further, the connecting portion 104 may be removably coupled to the pulse generator 102 so that the connecting portion 104 and the multi-electrode lead 106 may remain implanted during instances where the pulse generator 102 is removed from the body. For example, the pulse generator 102 may be temporarily removed to replace a power source 116 of the pulse generator 102. In some embodiments, the connecting portion 104 may be seamlessly integrated with the body 108, and the body 108 may be distinguished from the connecting portion 104 based on its intramyocardial position and/or the inclusion of the ring electrodes 110.

As will be further described herein, the multi-electrode lead 106 may be configured to be implanted via the coronary sinus vein. As such, the first end 107, the second end 109, and the wire outlet may be positioned proximate to the coronary sinus. However, it may be understood that the multi-electrode lead 106 may be implanted via other access points to the myocardium. Further, in some embodiments, the multi-electrode lead 106 may include a mechanically vulnerable breaking point 112 between where the multi-electrode lead 106 enters the myocardium and the blood space (e.g., proximate to the second end 109 of the intramyocardially positioned body 108). The breaking point 112 may enable the body 108 of the multi-electrode lead 106 to be disconnected from the remainder of the circumferential pacing system 100, including the connecting portion 104 and the pulse generator 102. As such, portions of the circumferential pacing system 100 positioned within the blood space (e.g., the connecting portion 104) may be withdrawn in the event of an associated infection without withdrawing the implanted body 108 of the multi-electrode lead 106.

The multi-electrode lead 106 may be adapted for an “over-the-wire” configuration for implantation. For example, the inner surface of the multi-electrode lead 106 may comprise a guidewire lumen for over-the-wire positioning. During implantation, a guidewire having an outer diameter that is smaller than the inner diameter of the multi-electrode lead 106 may be first navigated to a target position in the heart 101 (e.g., via the coronary sinus). For example, the guidewire may have an outer diameter of 0.014 inches, while the multi-electrode lead 106 may have an inner diameter of 0.0165 inches. The target position may include a pre-selected basal- or midventricular base-to-apex level of the left ventricle. In some embodiments, the target position may be proximate to a site of late electrical activation, or a site of late mechanical activation. In some embodiments, the target position may be proximate to the basal lateral left ventricle. The guidewire may be navigated via fluoroscopic, electroanatomic, and electrocardiographic depth navigation techniques. Once the guidewire is placed, the multi-electrode lead 106 may be advanced over the guidewire, such as pushed in place by a detachable catheter. As an example, lining the inner surface of the multi-electrode lead 106 with PTFE may reduce friction as the multi-electrode lead 106 is advanced over the guidewire. Additional structures of the multi-electrode lead 106 that may facilitate implantation are described herein with respect to FIGS. 7 and 8.

The pulse generator 102 further comprises the power source 116 and a control unit 118. The power source 116 may include a battery, such as a lithium iodide battery. In some examples, battery may be a rechargeable battery. In other examples, the power source 116 may not be rechargeable, and thus, the pulse generator 102 may be replaced in response to a low power condition. The pulse generator 102 may be a multi-channel generator. For example, one or a group (e.g., a portion) of the plurality of ring electrodes 110 may be coupled to a selected channel via the individual enamel-coated copper wire of each ring electrode. For example, a first group of the plurality of ring electrodes 110 may be coupled to a first channel of the pulse generator 102, a second group of the plurality of ring electrodes 110 may be coupled to a second channel of the pulse generator 102, a third group of the plurality of ring electrodes 110 may be coupled to a third channel of the pulse generator 102, and a fourth, remaining group of the plurality of ring electrodes 110 may be coupled to a fourth channel of the pulse generator 102. As a non-limiting example where there are 12 ring electrodes 110 and the pulse generator 102 has four channels, each of the four channels may be electrically coupled to 3 different ring electrodes. As another example, each of the ring electrodes 110 may be coupled to a different channel of the pulse generator 102. As still another example, only a subset of the ring electrodes may be electrically coupled to the pulse generator 102 depending on a desired pacing configuration of the circumferential pacing system 100. Thus, the pulse generator 102 may have more or fewer than four individuals pacing channels. For example, the pulse generator 102 may be a purpose-built generator for operating the multi-electrode lead 106. An output of each channel may be individually tuned during implantation based on a minimum capturing threshold for each ring electrode of the channel, for example.

The control unit 118 may comprise a computing device, such as a microcontroller. The control unit 118 may monitor the electrical activity of the heart 101 and analyze this activity in order to detect abnormalities in the morphology and/or timing in the electrical impulses in the heart 101. Based on the detected abnormalities, the pulse generator 102 may generate electrical impulses via electric circuitry and deliver the electrical impulses through the connecting portion 104 to one or more of the plurality of ring electrodes 110 via the enamel-coated copper wires. In some examples, the control unit 118 may adjust the current and/or voltage output by the pulse generator 102 for each channel. The control unit 118 may include a processor and a memory storing calibrations and computer-readable instructions (e.g., software) corresponding to one or more methods, an example of which is provided herein with respect to FIG. 11. For example, the processor may be configured to execute the computer-readable instructions stored in the memory in order to implement the one or more methods. In some examples, the control unit 118 may receive input from one or more sensors or sensing electrodes in order to monitor the mechanical and/or electrical activity of the heart 101. The sensors or sensing electrodes may be included in the pulse generator 102. Alternatively, the sensors or sensing electrodes may be external to the pulse generator 102 and may be in communication with the control unit 118.

When the multi-electrode lead 106 is implanted in the heart 101, the circumference of each of the ring electrodes 110 is completely surrounded by and in direct contact with the myocardium. As such, the plurality of ring electrodes 110 may deliver the electrical impulses generated by the pulse generator 102 directly to the surrounding myocardium, and the electrical impulses may further propagate through the myocardium to cause coordinated contraction via muscle depolarization and repolarization. In this way, the circumferential pacing system 100 may provide synchronous cardiac contraction.

In some embodiments, the circumferential pacing system 100 may be configured as a combined pacemaker and defibrillator (e.g., a CRT-D device). For example, the circumferential pacing system 100 may provide electrical impulses to the heart 101 to help the left and right ventricles contract synchronously and provide an electric shock in response to the control unit 118 detecting an abnormal heart rhythm (e.g., ventricular fibrillation). Additionally or alternatively, in some embodiments, the circumferential pacing system 100 may be configured to operate in atrioventricular pacing modes to allow and optimize atrioventricular pacing.

Turning now to FIGS. 2A and 2B, schematic cross-sectional views of a multi-electrode lead 206 positioned in a heart 201 are shown. The multi-electrode lead 206 may be the multi-electrode lead 106 of FIG. 1, for example. FIG. 2A depicts a short-axis view 200 of the heart 201, while FIG. 2B depicts a vertical long-axis view 250 of the heart 201. In particular, the short-axis view 200 depicts a cross-section of the left ventricle 202, the right ventricle 216, and the coronary sinus 218, while the vertical long-axis view 250 depicts a cross-section of the left ventricle 202 and the coronary sinus 218.

The heart 201 includes myocardium 204, which is bordered by epicardium 203 on the exterior and endocardium 205 on the interior. As can be seen in each of the short-axis view 200 and the vertical long-axis view 250, the multi-electrode lead 206 is positioned entirely within the myocardium 204. As particularly shown in the short-axis view 200 of FIG. 2A, the multi-electrode lead 206 encircles the left ventricle 202 and passes through IVS 220, which separates the left ventricle 202 and the right ventricle 216. Although FIG. 2A shows a circular shape for the multi-electrode lead 206, it may be understood that the multi-electrode lead 206 may have another generally curved shape that follows the curvature of the left ventricle 202 when implanted in the heart 201. Further, the curved shape may include a first end (or terminus) that first advances into the myocardium 204 during implantation and a second end (or terminus) at an exit site from the myocardium 204, such as depicted in FIG. 1 with respect to the multi-electrode lead 106. In some examples, the myocardium may be accessed via a shared entry site and exit site, while in other examples, the entry site and exit site may be at different locations. Because the multi-electrode lead 206 may be implanted via the coronary sinus 218, for example, the first and second ends may be positioned proximate to the coronary sinus 218. As such, the first end and the second end of the multi-electrode lead 206 may both be positioned within or proximate to the coronary sinus 218 or cardiac base. Alternatively, the entry and the exit site may be positioned at any arbitrary myocardial-blood interface in the walls of the left or right ventricle. For example, the myocardium may be accessed from the right ventricular surface of the interventricular septum and exited to the posterobasal left ventricle.

The multi-electrode lead 206 comprises a plurality of ring electrodes 210, which may be the plurality of ring electrodes 110 of FIG. 1 and may function as previously described. In the example shown in FIG. 2A, the multi-electrode lead 206 comprises 5 ring electrodes 210 having a variable inter-electrode spacing along a body 208 of the multi-electrode lead 206 (e.g., the body 108 of FIG. 1). The inter-electrode spacing is smaller at a first portion 212 of the multi-electrode lead 206 implanted in the anterior IVS 220 and larger at a second portion 214 of the multi-electrode lead 206 that is distal to the anterior IVS 220, such as near the posterior base of the heart 201. For example, the inter-electrode spacing has a first spacing s1 between adjacent ring electrodes 210 in the first portion 212 and second, larger spacing s2 between adjacent ring electrodes 210 in the second portion 214. The more concentrated ring electrode positioning at the IVS 220 may allow capture of proximal His branches to exploit a functional conduction system, if present, or synchronous basal activation if not present. However, it may be understood that other variations in the inter-electrode spacing are possible, including uniform spacing along the body 208 of the multi-electrode lead 206.

The arrangement of the ring electrodes 210 along the body 208 provides an array of electrodes around the myocardium 204, and the ring electrodes 210 may be configured in multiple different unipolar or bipolar configurations to overcome zones of poor excitation or high pacing thresholds in the heart 201. For example, the plurality of ring electrodes 210 may be divided into pairs for the bipolar pacing, with two immediately adjacent ring electrodes 210 forming each bipolar pacing pair. As another example, one or more common return electrodes may provide anodes for a plurality of cathodes for an at-a-distance bipolar configuration. Exemplary bipolar pacing pairs are shown in FIGS. 5 and 6. As another example, the ring electrodes 210 may be configured as multiple unipolar electrodes having the pulse generator as the anode. Anatomical sites in the heart 201 that may benefit from the specific positioning of individual ring electrodes 210, a higher density of the ring electrodes 210, and/or the positioning of common bipolar pacing return electrodes include the IVS 220 near the His bundle or left bundle branch or a position near the posterior base. Additionally, if only four ring electrodes 210 are available, it may be desirable to position one in the IVS 220, one in the posterior base, one in the lateral base, and one in the anterior base.

FIG. 2B shows one exemplary basal location of the multi-electrode lead 206, although other base-to-apex locations may be used. However, the basal location may enable the multi-electrode lead 206 to be positioned near the branching bundle of His for increased stimulation of the intrinsic cardiac conduction system, as will be elaborated below with respect to FIG. 4.

Turning now to FIG. 3, a schematic vertical long-axis view 300 of the left ventricle 202 of the heart 201 is shown. As such, components previously introduced in FIGS. 2A and 2B are numbered the same and will not be reintroduced. The vertical long-axis view 300 of FIG. 3 schematically depicts the propagation of electrical impulses 302 delivered by the plurality of ring electrodes 210 through activation pathways in the myocardium 204. In the example shown in FIG. 3, the myocardium 204 includes a scar 304, which impedes propagation of the electrical impulses 302. However, because the multi-electrode lead 206 exploits all basal pacing sites simultaneously, the scar 304 interrupts only a fraction of the activation pathways. As such, the multi-electrode lead 206 effectively provides circumferential multi-polar base-to-apex pacing throughout the left ventricle 202 even in the presence of the scar 304. It may be understood that the density of the ring electrodes 210 shown in FIG. 3 is exemplary, and the density of the ring electrodes 210 and/or pacing channels may be varied as desired.

Because the pacing sites (e.g., the ring electrodes 210) are positioned intramyocardially, the pacing sites are not subject to dislodgement. Further, because of the intramyocardial position of the ring electrodes 210, the ring electrodes 210 make uniform circumferential contact with the myocardium 204, eliminating the issue of variable contact that may occur with typical endocardial transvenous pacing electrodes or epicardial pacing electrodes and eliminating the risk of dislodgement. For example, the completely intramyocardial ring electrodes 210 may have increased contact with the myocardium 204 compared with traditional endocardial or epicardial pacing electrodes. As used herein, “circumferential contact” refers to an outer surface defining the circumference of a given ring electrode being in direct contact with the surrounding myocardium.

As mentioned above, the multi-electrode lead 206 may be used for direct His bundle or left bundle branch (LBB) pacing (LBBP). Turning to FIG. 4, a schematic view 400 shows placement of the multi-electrode lead 206 within the IVS 220 with respect to the cardiac conduction system (e.g., the ventricular septal conduction system). A dashed line 402 indicates separation between the atria and ventricles. When cardiac conduction is functional, an atrioventricular node (AVN) 404 receives a pacemaking signal that originates at the sinoatrial node from the right atrium and transmits it along His bundle (HB) 406 and through left bundle branch (LBB) 408 and right bundle branch (RBB) 410 to cause coordinated (e.g., synchronous) contraction of the ventricles. However, cardiac conduction abnormalities caused by, for example, dispersion of activation or variation in refractoriness may lead to cardiac dyssynchrony. For example, LBBB may cause delayed contraction of the left ventricle due to slow or absent conduction through the LBB 408, which may result in adverse ventricular remodeling and heart failure.

As such, the multi-electrode lead 206 may be placed in the IVS 220 proximate to the LBB 408 and the HB 406 to stimulate the cardiac conduction system via LBB or HB pacing. Additionally or alternatively, the multi-electrode lead 206 may directly activate the IVS 220 to provide synchronous pacing. This synchronous pacing may provide increased positive patient outcomes compared with right ventricular pacing, for example. Further, the intramyocardial position within the IVS 220 and basal location of the ring electrodes 210 may reduce interference with tricuspid valve leaflet integrity and reduce excursion while also avoiding non-target depolarization, such as undesirable phrenic nerve stimulation.

Referring now to FIG. 5, a schematic cross-sectional view of a multi-electrode lead 506 positioned in the heart 201 is shown. The multi-electrode lead 506 may be the multi-electrode lead 106 of FIG. 1, for example. The view shown in FIG. 5 is similar to the short-axis view 200 of the multi-electrode lead 206 shown in FIG. 2A, and the position of the multi-electrode lead 506 in the heart 201 is described above with respect to the multi-electrode lead 206. Further, the multi-electrode lead 506 functions similarly to the multi-electrode lead 206 of FIGS. 2A and 2B, with like components numbered similarly. As such, for brevity, the differences between the multi-electrode lead 506 and the multi-electrode lead 206 will be discussed herein.

In the example shown in FIG. 5, the multi-electrode lead 506 comprises 9 ring electrodes 510, although a different number of ring electrodes 510 may be included in other examples (e.g., 2-8 ring electrodes or more than 9 ring electrodes). In the present example, the ring electrodes 510 comprise a first ring electrode 510a, a second ring electrode 510b, a third ring electrode 510c, a fourth ring electrode 510d, a fifth ring electrode 510e, a sixth ring electrode 510f, a seventh ring electrode 510g, an eighth ring electrode 510h, and a ninth ring electrode 510i. The multi-electrode lead 506 is operated in a first bipolar pacing configuration 500, where the second ring electrode 510b and the third ring electrode 510c comprise a first bipolar pair 522 and the sixth ring electrode 510f and the seventh ring electrode 510g comprise a second bipolar pair 524. The second ring electrode 510b comprises the cathode and the third ring electrode 510c comprises the anode in the first bipolar pair 522. During operation, the second ring electrode 510b may receive an electrical impulse from a connected pulse generator (e.g., the pulse generator 102 of FIG. 1), and electric current flows from the second ring electrode 510b to the third ring electrode 510c via the myocardium 204 in response to the electrical impulse. Similarly, the sixth ring electrode 510f comprises the cathode and the seventh ring electrode 510g comprises the anode in the second bipolar pair 524, and thus, electric current flows from the sixth ring electrode 510f to the seventh ring electrode 510g via the myocardium 204 in response to the sixth ring electrode 510f receiving the electrical impulse from the pulse generator. However, in other examples, the anode and cathode may be exchanged such that the second ring electrode 510b comprises the anode and the third ring electrode 510c comprises the cathode in the first bipolar pair 522 and the sixth ring electrode 510f comprises the anode and the seventh ring electrode 510g comprises the cathode in the second bipolar pair 524.

The additional ring electrodes 510 that are not included in the first bipolar pair 522 and the second bipolar pair 524 (e.g., the first ring electrode 510a, the fourth ring electrode 510d, the fifth ring electrode 510e, the eighth ring electrode 510h, and the ninth ring electrode 510i) may not be operated (e.g., may not receive electric current from the pulse generator) while the multi-electrode lead 506 is operated in the first bipolar pacing configuration 500, at least in some embodiments. For example, a portion of the electrodes may be disabled to conserve battery life via pulse generator programming or using an adapter to select which electrodes are physically connected to the pulse generator when fewer connectors are available on the pulse generator than electrodes. However, in other embodiments, all of the ring electrodes 510 may be operated.

FIG. 6 shows a schematic cross-sectional view of the multi-electrode lead 506 in a second bipolar pacing configuration 600. As such, components are numbered the same and will not be re-introduced, with differences between the first bipolar pacing configuration 500 of FIG. 5 and the second bipolar pacing configuration 600 of FIG. 6 described herein.

In the second bipolar pacing configuration 600, the third ring electrode 510c and the fourth ring electrode 510d comprise a first bipolar pair 622, and the eighth ring electrode 510h and the ninth ring electrode 510i comprise a second bipolar pair 624. The third ring electrode 510c comprises the cathode and the fourth ring electrode 510d comprises the anode in the first bipolar pair 622. During operation, the third ring electrode 510c may receive an electrical impulse from the pulse generator, and electric current flows from the third ring electrode 510c to the fourth ring electrode 510d via the myocardium 204 in response to the electrical impulse. Similarly, the eighth ring electrode 510h comprises the cathode and the ninth ring electrode 510i comprises the anode in the second bipolar pair 624, and thus, electric current flows from the eighth ring electrode 510h to the ninth ring electrode 510i via the myocardium 204 in response to the eighth ring electrode 510h receiving the electrical impulse from the pulse generator. However, in other embodiments, third ring electrode 510c comprises the anode and the fourth ring electrode 510d comprises the cathode in the first bipolar pair 622, and the eighth ring electrode 510h may comprise the anode and the ninth ring electrode 510i may comprise the cathode in the second bipolar pair 624.

As such, the multi-electrode lead 506 may be adapted for multiple different bipolar and unipolar pacing configurations as desired by differently operating particular ring electrodes 510, such as based on their positioning in the heart 201 and the desired pacing configuration. In particular, providing the bipolar pacing near the IVS and also near the posterior base of the left ventricle in each of the first bipolar pacing configuration 500 and the second bipolar pacing configuration 600 may provide effective His bundle and LBB pacing. In this way, the multi-electrode lead 506 provides a versatile intramyocardial pacing device.

Turning to FIG. 7, a rendering 700 of a first exemplary embodiment of a synchronous circumferential pacing (SCIRP) lead 706 is shown. The SCIRP lead 706 is one example of the multi-electrode lead 106 of FIG. 1, the multi-electrode lead 206 of FIGS. 2A-4, or the multi-electrode lead 506 of FIGS. 5 and 6 and may function as previously described. It may be understood that the relative dimensions shown in FIG. 7 are exemplary and variations may be used without departing from the scope of this disclosure.

The SCIRP lead 706 includes a body 708 having 12 ring electrodes 710 distributed along its length, which extends from a first end 707 to a second end 709. However, it may be understood that the SCIRP lead 706 may have fewer ring electrodes 710, such as in a range from 4-6. The body 708 may have a length of approximately 300 millimeters (mm). For example, the body 708 may have a length that is in a range from 250-350 mm, although other lengths are possible. The length of the body 708 may be sized to approximately encircle the left ventricle of the heart.

In the first exemplary embodiment, each ring electrode 710 has a length of 0.50 mm with uniform inter-electrode spacing between adjacent ring electrodes 710. For example, the inter-electrode spacing between adjacent ring electrodes may be in a range between 1 and 200 mm, although other spacings are possible. The inter-electrode spacing between adjacent electrodes may vary based on the length of the body 708, the number of ring electrodes 710 included in the SCIRP lead 706, and whether the ring electrodes 710 are variably or uniformly spaced.

The SCIRP lead 706 includes a tapered nose cone 711 that extends from a first ring electrode 710a to the first end 707. In the present example, the tapered nose cone 711 has a length that is approximately one-tenth of the length of the body 708. As a non-limiting example, the length of the tapered nose cone 711 may be approximately 20 mm. The tapered nose cone 711 is a region where the outer diameter of the body 708 gradually decreases in order to reduce a diameter mismatch between the SCIRP lead 706 and a guidewire used during implantation, which may help ease the implantation of the SCIRP lead 706 in the myocardium. For example, the tapered nose cone 711 may advance within the myocardium from an entry site from the coronary sinus via pushing and/or pulling. Due to the tapered nose cone 711, the first end 707 has a smaller outer diameter d1, while the second end 709 has a larger outer diameter d2. For example, the SCIRP lead 706 may have a uniform diameter (e.g., d2) from the first ring electrode 710a to the second end 709, and the tapered nose cone 711 may taper in diameter from d2 to d1. As non-limiting examples, d1 may be 0.89 mm and d2 may be 1.30 mm. In other embodiments, the length of the SCIRP lead may be tapered so that the lead gradually (e.g., continuously) or abruptly tapers (e.g., in a step-wise fashion) toward a minimum tip diameter from any point along its length (e.g., any point intermediate the first end 707 and the second end 709). In association with this taper, the plurality of ring electrodes 710 may have variable diameters and surface areas, with larger diameters and surface areas where the diameter of the SCIRP lead 706 is larger and gradually decreasing diameters and surface areas as the diameter of the SCIRP lead 706 decreases toward the minimum tip diameter at the first end 707. In some embodiments, the taper may be uniformly continued from the second end 709 to the first end 707. In such embodiments, electrode diameters are matched based on their location in the taper, being larger proximal to the second end 709 and narrower distal to the second end 709. As such, a least a portion of the SCIRP lead 706 may have a decreasing diameter toward the first end 707, which is configured to be inserted into the myocardium before the second end 709 during implantation.

Although not shown in FIG. 7, in some embodiments, the SCIRP lead 706 may further include a mechanical ring (e.g., loop) or other detachable pull mechanism positioned on the tapered nose cone 711 that is adapted to receive a removable tether. For example, the tether may be removably attached to the mechanical ring in order to pull the tapered nose cone 711 of the SCIRP lead 706 antegrade in a veno-venous or veno-cameral fashion during implantation. For example, the detachable pull mechanism may be a looped suture or microthin wire including a detachable screw or lock to allow traction on a removable leader along a trajectory of a guidewire during implantation. Thus, the SCIRP lead 706 may be simultaneously pulled at the first end 707 via the tethered mechanical ring and pushed at the second end 709 during implantation. After the SCIRP lead 706 reaches the desired position within the heart muscle, the tether may be removed from the mechanical ring of the tapered nose cone 711, for example.

The tapered nose cone 711 may have a greater length than a rear portion of the SCIRP lead 706 between a final ring electrode 710b and the second end 709, at least in some embodiments. Thus, the larger length of the tapered nose cone 711 may enable a more gradual change in diameter for smoother insertion.

FIG. 8 shows a rendering 800 of a second exemplary embodiment of a SCIRP lead 806. The SCIRP lead 806 is one example of the multi-electrode lead 106 of FIG. 1, the multi-electrode lead 206 of FIGS. 2A-4, or the multi-electrode lead 506 of FIGS. 5 and 6 and may function as previously described. It may be understood that the relative dimensions shown in FIG. 8 are exemplary and variations may be used without departing from the scope of this disclosure.

The SCIRP lead 806 includes a body 808 having twelve ring electrodes 810 distributed along its length, which extends from a first end 807 to a second end 809. However, it may be understood that the SCIRP lead 806 may have fewer ring electrodes 810, such as in a range from 4-6. The SCIRP lead 806 is similar to the SCIRP lead 706 of FIG. 7, and as such, the present description will describe the differences between the SCIRP lead 806 and the SCIRP lead 706. In the second exemplary embodiment, each ring electrode 810 has a length of 0.10 mm with a uniform inter-electrode spacing between adjacent ring electrodes 810. Due to the smaller length of the ring electrodes 810 compared with the ring electrodes 710 of FIG. 7, the inter-electrode spacing between adjacent ring electrodes may be larger than in FIG. 7.

The SCIRP lead 806 includes a tapered nose cone 811 that extends from a first ring electrode 810a to the first end 807. In the present example, the tapered nose cone 811 has the same dimensions as the tapered nose cone 711 of FIG. 7 and functions as previously described. For example, the tapered nose cone 811 may also optionally include a mechanical ring for tethering during insertion. Further, the diameter of the body 808 of the SCIRP lead 806 from the first ring electrode 810a to the second end 809 is the same as in the SCIRP lead 706 of FIG. 7.

Both of the first exemplary embodiment of the SCIRP lead shown in FIG. 7 and the second exemplary embodiment of the SCIRP lead shown in FIG. 8 may enable increased electrode-tissue impedance due to the ultra-narrow ring electrodes of less than 1 mm in length (e.g., 0.50 mm in FIGS. 7 and 0.1 mm in FIG. 8). For example, the decreased surface area of the ultra-narrow ring electrodes of the SCIRP lead results in lower surface contact area, which further results in increased resistance. As one example, the ring electrodes of the SCIRP lead may each have a tissue contact impedance of greater than 1 ohm. As another example, each of the electrodes of the SCIRP lead may have a tissue contact impedance of greater than 20 ohms.

The intramyocardial electrodes described herein have increased load resistance compared with standard right ventricle or left ventricle endovenous endocardial electrodes by minimizing the low-resistance blood path, as standard transvenous electrodes are exposed to blood and have variable contact with the myocardium. The smaller intramyocardial electrodes increase impedance, which is desirable to limit current and increase a voltage drop at the electrode interface with the myocardium. The reduced current draw increases pulse generator longevity. Intramyocardial electrodes also avoid the endovascular lead issue of wasted parallel voltage drop from the electrode to the blood pool, and this avoidance reduces the current draw for myocardial capture for an intramyocardial electrode.

Due to this increased electrode-tissue impedance, battery depletion by the SCIRP lead may be decreased compared with conventional endovenous pacemaker leads that have obligate blood pool exposure. For example, as explained with respect to FIG. 1, the SCIRP lead may be included in a pacing system that includes a pulse generator having a power source (e.g., the power source 116 of FIG. 1), and the lower battery depletion may enable less frequent recharging or replacement of the power source. Further, for a given battery energy budget, the intramyocardial electrode placement of the ring electrodes of the SCIRP lead allow more channels to be used, which may decrease battery life compared to operating fewer channels but may enable more channels to be used compared with conventional endovenous pacemaker leads.

Next, FIG. 9 shows an example method 900 for implanting a SCIRP lead (e.g., the multi-electrode lead 106) within myocardium encircling the left ventricle of a heart of a subject.

At 902, the method 900 includes advancing a guidewire into the myocardium via transvascular entry. For example, the guidewire may traverse the myocardium via the circulatory system of the subject and allow a lumen-containing SCIRP lead to be advanced into a myocardial position. In particular, the guidewire may access the coronary sinus via the venae cavae and exit the coronary sinus into the ventricular myocardium for a transvenous entry. However, in other examples, the guidewire may advance into the myocardium via transarterial entry.

At 904, the method 900 includes navigating the guidewire though the myocardium at a desired base-to-apex level to define a trajectory for SCIRP lead implantation. For example, an operator may navigate the guidewire via fluoroscopy (e.g., biplane fluoroscopy), intracardiac electrograms, and ultrasonography. The fluoroscopy and ultrasonography (e.g., cardiac ultrasound) may provide a visual indication of the guidewire within the heart, while the intracardiac electrograms may indicate a radial depth of the guidewire within the ventricular myocardium (e.g., a depth from the epicardial and endocardial borders of the myocardium). The guidewire may traverse some or all of the trajectory for SCIRP lead implantation before entering a cardiac chamber where it can be advanced for guidewire purchase or ensnared to apply counter traction or externalization, as will be elaborated below at 906. The desired base-to-apex level may be a pre-selected basal implantation level, for example, in order to position the SCIRP lead proximate cardiac conduction system structures, such as the His bundle.

In some embodiments, the method 900 includes techniques to accomplish strong guidewire “purchase” to facilitate advancing the SCIRP lead over the guidewire. These techniques include advancing the guidewire deep into the myocardium or navigating the guidewire deep into a heart chamber such as the right ventricle or left ventricle where it optionally may be ensnared, as optionally indicated at 906. For example, the guidewire may be ensnared by an antegrade transvenous snare catheter (e.g., having a same direction of guidewire advancement) or a retrograde arterial transaortic snare catheter (e.g., having the opposite direction to the direction of guidewire advancement around the ventricle). The snare catheter may include one or more loops that capture a distal end of the guidewire to optionally provide counter-traction. The guidewire may traverse some or all of the trajectory for the SCIRP lead implantation before ensnarement.

At 908, the method 900 includes advancing a SCIRP lead delivery system into the myocardium along the guidewire. The SCIRP lead delivery system may be advanced along the guidewire-defined trajectory by sliding the SCIRP lead over the guidewire, with a leading, distal end of the SCIRP lead including a smallest diameter portion of the SCIRP lead in order to reduce a diameter mismatch between the guidewire and the SCIRP lead to ease implantation, such as described above with respect to FIGS. 7 and 8. For example, a catheter may be used to push a second end of the SCIRP lead that is opposite to the smallest diameter portion. The smallest diameter portion may include a tapered nose cone, for example, or the SCIRP lead may be tapered along its length in a continuous taper or step-wise taper. For example, the diameter of the SCIRP lead may gradually increase from the distal end of the SCIRP lead to a largest diameter portion, which may be at the second end of the SCIRP lead and/or an intermediate portion of the SCIRP lead between the distal end and the second end.

At 910, the method 900 optionally includes ensnaring a pull mechanism of the SCIRP lead delivery system via a retrograde leader. The pull mechanism may be a mechanical ring, a loop, a hook, or another structure that can be captured by the retrograde leader. The retrograde leader may be a looped suture or microthin wire, for example, including a detachable screw or lock to allow traction on the pull mechanism. Once the pull mechanism is captured, the SCIRP lead may be additionally pulled antegrade along the guidewire-defined trajectory via the retrograde leader, if desired.

At 912, the method 900 includes pushing and/or pulling the SCIRP lead to advance the SCIRP lead along the guidewire-defined trajectory within the myocardium. For example, fluoroscopy may be used to visually monitor the position of the SCIRP lead along the guidewire-defined trajectory, and the SCIRP lead may be advanced until the SCIRP lead encircles a desired portion of the circumference of the left ventricle.

At 914, the method 900 includes evaluating the rotational circumferential position of ring electrodes of the SCIRP lead. For example, once the SCIRP lead encircles the desired portion of the circumference of the left ventricle, the rotational circumferential position of the ring electrodes may be fine-tuned in order to achieve a desired position that advantageously positions pre-determined ring electrodes with respect to anatomical features. As one example, a pre-determined ring electrode on the SCIRP lead may have a desired rotational circumferential position around the left posterior fascicle of the LBB or bundle of His, close to the left ventricle endocardial surface around the left ventricle septal myocardium, in order to facilitate His bundle or LBB pacing. As an example, the rotational circumferential position of the pre-determined ring electrode with respect to the LBB or bundle of His may be determined via intracardiac electrogram sensing or pacing to check left ventricle activation times, which may be measured by a surface ECG or by other SCIRP electrodes. For example, it may be determined that the pre-determined ring electrode is in the desired position in response to the left ventricle activation time being less than a non-zero, pre-determined threshold time. As another example, if the ring electrodes are variably spaced along the body of the SCIRP lead, the desired rotational circumferential position may include positioning the ring electrodes with a higher concentration (e.g., density) at the intraventricular septum and posterior base of the left ventricle.

At 916, the method 900 includes determining if the desired position has been achieved. If the desired position has not yet been achieved (e.g., the left ventricle activation time is greater than or equal to the pre-determined threshold time), the method 900 proceeds to 918 and includes pushing and/or pulling the SCIRP lead to adjust the rotational circumferential positioning of the SCIRP lead. The method 900 may then return to 914 to re-evaluate the position. In some embodiments, the rotational circumferential positioning may be incrementally adjusted in between evaluating the position, while in other embodiments, the rotational circumferential position of the SCIRP lead may be adjusted while the positioning is evaluated (e.g., substantially at the same time). If adequate capture is not achieved by rotational positioning (such as in the presence of an extensive basal scar), then the method 900 may return to 904 and reposition electrodes in a more apical location distal to the basal scar, as detected by electrograms or imaging.

Returning to 916, if the desired positioning has been achieved, the method 900 proceeds to 920 and includes withdrawing the SCIRP lead delivery system, including any catheters or leaders (e.g., pull structures), leaving the SCIRP lead in position. Thus, the SCIRP lead may remain implanted within the myocardium surrounding the left ventricle while components used in the implantation process may be removed from the subject's body. Further, the ring electrodes of the SCIRP lead may be evaluated for pacing capture thresholds, which may be used to program a pulse generator electrically coupled to the ring electrodes. The method 900 may then end.

FIG. 10 shows an example method 1000 for setting up electrodes of a SCIRP lead (e.g., the multi-electrode lead 106) that has been implanted within myocardium encircling the left ventricle of a heart of a subject. Electrode optimization according to the method 1000 may help ensure that electrical impulses from the intramyocardial electrodes of the SCIRP lead (e.g., the ring electrodes 110) stimulate the myocardium and/or cadiac conduction system without expending excess energy in order to prolong pulse generator battery life.

At 1002, the method 1000 includes determining capture threshold for each electrode pair (e.g., unipolar and bipolar pairs).

At 1004, the method 1000 include rejecting electrode(s) with capture threshold(s) over an upper threshold from further optimization. The upper threshold may be a pre-determined non-zero capture threshold that distinguishes electrodes able to generate propagating contraction in the heart from those that do not reliably produce a contractile response.

At 1006, the method 1000 includes evaluating conduction system capture based on an R wave peak time. The R wave peak time may refer to a pace to lateral precordial lead R wave peak time. The R wave peak time may be used to determine if adequate threshold conduction system capture has been achieved.

At 1008, the method 1000 includes determining if the R wave peak time is less than a threshold. The threshold may be a pre-determined (e.g., pre-specified) non-zero time duration.

If the R wave peak time is less than the threshold, the method 1000 proceeds to 1010 and includes performing further testing during baseline rhythm.

If the R wave peak time is not less than the threshold (e.g., the R wave peak time is greater than or equal to the threshold), the method 1000 proceeds to 1012 and includes performing further testing during conduction system pacing.

At 1014, the method 1000 includes determining a latest (e.g., most delayed with respect to time) activated electrode by electrical or mechanical activation.

At 1016, the method 1000 includes determining if the latest activation exceeds a delay threshold. The delay threshold may be a pre-determined, non-zero time duration that is different than the time duration of the R wave peak time threshold, for example.

If the latest activation does not exceed the delay threshold, the method 1000 proceeds to 1024 and includes ending the electrode optimization process. The method 1000 ends accordingly.

If the latest activation exceeds the delay threshold, the method 1000 proceeds to 1018 and includes selecting an optimal threshold configuration for the electrode at the latest activated site.

At 1020, the method 1000 includes performing further testing with the most delayed activated electrode added to the pacing configuration during late activation assessment.

At 1022, the method 1000 includes determining if there are additional electrodes to test. If there are additional electrodes to test, the method 1000 returns to 1014 to again determine the most delayed activated electrode by electrical or mechanical activation. If there are not additional electrodes to test, the method 1000 proceeds to 1024 to end the electrode optimization process.

FIG. 11 shows a flow-chart of an example method 1100 for operating a pacing system including an intramyocardial multi-electrode lead that encircles the left ventricle of a heart of a subject. The method 1100 is described with regard to the systems and components of FIGS. 1-8, though it should be appreciated that the method 1000 may be implemented with other systems and components without departing from the scope of the present disclosure. The method 1100 may be carried out according to instructions stored in non-transitory memory of a computing device of the pacing system, such as the control unit 118 of the pulse generator 102 of FIG. 1. Although the method 1100 will be described with respect to cardiac resynchronization therapy (CRT), it may be understood that the method 1100 may be adapted for other types of pacing.

At 1102, the method 1100 includes generating electrical impulses via the pulse generator. Generating the electrical impulses via the pulse generator may include determining a current and/or voltage for each channel of the pulse generator based on a channel output tuning and a type of pacing that is to be provided (e.g., unipolar or bipolar pacing), as indicated at 1104. For example, the channel output tuning may be performed during implantation by an operator measuring a minimum capturing threshold of each ring electrode and stored in the memory so that the pulse generator is programmed to adjust the output current/voltage for each channel accordingly. As discussed above with respect to FIG. 1, the channels may be connected to individual ring electrodes or groups of ring electrodes depending on a number of available channels versus ring electrodes and/or a desired pacing configuration.

At 1106, the method 1100 includes transmitting the generated electrical impulses from each channel of the pulse generator to the corresponding ring electrodes of the intramyocardial multi-electrode lead. For example, the pulse generator may be implanted within the subject and electrically connected to the ring electrodes via copper wires encased within insulation (e.g., the body 108 and connecting portion 104 of the multi-electrode lead 106 FIG. 1). As such, the electrical impulse generated by each channel of the pulse generator may travel from a space external to the heart to the intramyocardial space within the heart. The pulse generator may transmit the electrical impulses via a single channel or a plurality of channels.

At 1108, the method 1100 includes applying the electrical impulses directly to the myocardium of the left ventricular wall via the ring electrodes having circumferential contact with the myocardium. In some examples, the method 1100 may further include stimulating the cardiac conduction system via the electrical impulses applied directly to the myocardium via the ring electrodes, as optionally indicated at 1110. For example, as explained above with respect to FIG. 4, the intramyocardial multi-electrode lead may traverse the intraventricular septum at a basal position that is proximate to the His bundle and the LBB. As another example, the intramyocardial multi-electrode lead may be embedded wholly or partially in the myocardium of a left ventricular wall. As another example, the intramyocardial multi-electrode lead may be embedded in a His bundle branch. As another example, the intramyocardial multi-electrode lead may be positioned proximate a left posterior fascicular conduction system. As such, the electrical impulses may stimulate at least a portion of the cardiac conduction system, such as the ventricular septal conduction system, and/or directly activate contractile tissue of the heart (e.g., the contractile tissue in the intraventricular septum). In one example, electrical impulses may be applied directly to a portion of the cardiac conduction system comprising the His bundle. In another example, electrical impulses may be applied directly to the portion of the cardiac conduction system comprising the left bundle branch conduction system. The method 1100 may then return so that subsequent electrical pulses may be generated.

In this way, SCIRP leads may be employed for CRT with a higher success rate (e.g., lower non-responder rate). Furthermore, the SCIRP leads may be used for physiological (His bundle and LBB) pacemaker therapy with a lower rate of lead dislodgement and tricuspid valve injury compared with conventional CRT approaches. By utilizing intramyocardial positioning for the pacemaker ring electrodes, the pacemaker ring electrodes have increased contact with the myocardial wall, while the ultra-narrow length of each electrode may help reduce pacemaker battery usage to increase battery life. Furthermore, the intramyocardial deployment avoids non-target depolarization, such as undesirable phrenic nerve stimulation. Overall, positive patient outcomes may be increased.

The technical effect of providing synchronous circumferential pacing throughout the left ventricle of the heart via an intramyocardial multi-electrode lead is that cardiac resynchronization may be achieved with increased homogeneity, reduced lead dislodgement, and reduced battery depletion.

The disclosure also provides support for an apparatus for a multi-electrode pacemaker lead, comprising: a plurality of ring electrodes distributed along a length of the multi-electrode pacemaker lead, the multi-electrode pacemaker lead configured to be implanted within myocardium of a wall of a ventricle of a heart such that an outer surface defining a circumference of at least one of the plurality of ring electrodes is in direct contact with the myocardium. In a first example of the system, the multi-electrode pacemaker lead is shaped to extend around at least 180 degrees of a ventricular circumference of a left ventricle when implanted within the myocardium of the wall of the ventricle of the heart. In a second example of the system, optionally including the first example, each of the plurality of ring electrodes comprises a cylindrical shape having length in a dimension parallel to a central axis of the cylindrical shape, and wherein the length is 1 millimeter or less. In a third example of the system, optionally including one or both of the first and second examples, the length of one or more of the plurality of ring electrodes is 0.1 millimeters or less. In a fourth example of the system, optionally including one or more or each of the first through third examples, the multi-electrode pacemaker lead is configured for an over-the-wire configuration for implantation within the myocardium of the wall of the ventricle of the heart. In a fifth example of the system, optionally including one or more or each of the first through fourth examples, the multi-electrode pacemaker lead comprises variable inter-electrode spacing between the plurality of ring electrodes along the length of the multi-electrode pacemaker lead, and wherein a spacing distance between each of the plurality of ring electrodes along the length of the multi-electrode pacemaker lead ranges from 1 millimeter and 200 millimeters. In a sixth example of the system, optionally including one or more or each of the first through fifth examples, the multi-electrode pacemaker lead is configured to be positioned within the myocardium of the left ventricle of the heart such that the variable inter-electrode spacing is smaller in a portion proximate to at least one of an interventricular septum in a region of a His bundle, or a left posterior fascicular conduction system, or a basal lateral left ventricle, or a site of late electrical activation, or a site of late mechanical activation. In a seventh example of the system, optionally including one or more or each of the first through sixth examples, the plurality of ring electrodes comprises ring electrodes having varying lengths, and wherein multi-electrode pacemaker lead is implanted to position a pre-determined one of the plurality of ring electrodes at a desired rotational position around the ventricle of the heart. In a eighth example of the system, optionally including one or more or each of the first through seventh examples, the variable inter-electrode spacing between the plurality of ring electrodes is configured to generate intramyocardial unipolar pacing at one or multiple of the plurality of ring electrodes or bipolar pacing between at least one cathode of the plurality of ring electrodes and at least one anode of the plurality of ring electrodes, and wherein a maximum spacing distance between each of the plurality of ring electrodes along the length of the multi-electrode pacemaker lead is equal to a minimum spacing distance multiplied by one less than a number of ring electrodes in the plurality of ring electrodes. In a ninth example of the system, optionally including one or more or each of the first through eighth examples, the multi-electrode pacemaker lead comprises a tapered nose cone on a first end, or wherein at least a portion of the length of the multi-electrode pacemaker lead is tapered toward the first end, with a diameter decreasing toward an end of the multi-electrode pacemaker lead that is configured to be inserted into the myocardium first during implantation. In a tenth example of the system, optionally including one or more or each of the first through ninth examples, the multi-electrode pacemaker lead comprises a detachable pull mechanism for implantation, and wherein the detachable pull mechanism is adapted to receive a retrograde tether configured to pull the multi-electrode pacemaker lead antegrade in during implantation of the multi-electrode pacemaker lead within the myocardium of the wall of the ventricle the heart. In a eleventh example of the system, optionally including one or more or each of the first through tenth examples, the multi-electrode pacemaker lead includes a fracture site that is configured to be positioned at a myocardial-blood-space interface when implanted within the myocardium of the wall of the ventricle of the heart.

The disclosure also provides support for a method, comprising: generating electrical impulses via a pulse generator, transmitting the electrical impulses from the pulse generator to a plurality of ring electrodes of a pacing lead embedded within myocardium of a heart via a single channel or plurality of channels, and applying the electrical impulses directly to the myocardium via the plurality of ring electrodes, wherein at least one of the plurality of ring electrodes is in circumferential contact with the myocardium. In a first example of the method, the pacing lead is embedded wholly or partially within the myocardium of a left ventricular wall or interventricular septum in proximity to a conduction system of the heart, and wherein applying the electrical impulses directly to the myocardium via the plurality of ring electrodes comprises applying the electrical impulses directly to the myocardium of the left ventricular wall of the heart. In a second example of the method, optionally including the first example, the pacing lead encircles at least 180 degrees around a base of a left ventricle of the heart, and wherein applying the electrical impulses directly to the myocardium via the plurality of ring electrodes comprises applying base-to-apex pacing throughout an entirety of the left ventricle. In a third example of the method, optionally including one or both of the first and second examples, generating the electrical impulses via the pulse generator comprises tuning each of the electrical impulses based on a channel of the pulse generator, a channel output tuning stored in memory, and a desired type of pacing. In a fourth example of the method, optionally including one or more or each of the first through third examples, the desired type of pacing is unipolar pacing or bipolar pacing. In a fifth example of the method, optionally including one or more or each of the first through fourth examples, a portion of the pacing lead is embedded within the myocardium of an intraventricular septum of the heart, and the method further comprises applying the electrical impulses directly to a portion of a cardiac conduction system of the heart, and wherein the portion of the cardiac conduction system comprises a His bundle or wherein the portion of the cardiac conduction system comprises a left bundle branch conduction system.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An apparatus for a multi-electrode pacemaker lead, comprising:
a plurality of ring electrodes distributed along a length of the multi-electrode pacemaker lead, the multi-electrode pacemaker lead configured to be implanted within myocardium of a wall of a ventricle of a heart such that an outer surface defining a circumference of at least one of the plurality of ring electrodes is in direct contact with the myocardium.

2. The apparatus of claim 1, wherein the multi-electrode pacemaker lead is shaped to extend around at least 180 degrees of a ventricular circumference of a left ventricle when implanted within the myocardium of the wall of the ventricle of the heart.

3. The apparatus of claim 2, wherein each of the plurality of ring electrodes comprises a cylindrical shape having length in a dimension parallel to a central axis of the cylindrical shape, and wherein the length is 1 millimeter or less.

4. The apparatus of claim 3, wherein the length of one or more of the plurality of ring electrodes is 0.1 millimeters or less.

5. The apparatus of claim 1, wherein the multi-electrode pacemaker lead is configured for an over-the-wire configuration for implantation within the myocardium of the wall of the ventricle of the heart.

6. The apparatus of claim 2, wherein the multi-electrode pacemaker lead comprises variable inter-electrode spacing between the plurality of ring electrodes along the length of the multi-electrode pacemaker lead, and wherein a spacing distance between each of the plurality of ring electrodes along the length of the multi-electrode pacemaker lead ranges from 1 millimeter and 200 millimeters.

7. The apparatus of claim 6, wherein the multi-electrode pacemaker lead is configured to be positioned within the myocardium of the left ventricle of the heart such that the variable inter-electrode spacing is smaller in a portion proximate to at least one of an interventricular septum in a region of a His bundle, or a left posterior fascicular conduction system, or a basal lateral left ventricle, or a site of late electrical activation, or a site of late mechanical activation.

8. The apparatus of claim 6, wherein the plurality of ring electrodes comprises ring electrodes having varying lengths, and wherein multi-electrode pacemaker lead is implanted to position a pre-determined one of the plurality of ring electrodes at a desired rotational position around the ventricle of the heart.

9. The apparatus of claim 6, wherein the variable inter-electrode spacing between the plurality of ring electrodes is configured to generate intramyocardial unipolar pacing at one or multiple of the plurality of ring electrodes or bipolar pacing between at least one cathode of the plurality of ring electrodes and at least one anode of the plurality of ring electrodes, and wherein a maximum spacing distance between each of the plurality of ring electrodes along the length of the multi-electrode pacemaker lead is equal to a minimum spacing distance multiplied by one less than a number of ring electrodes in the plurality of ring electrodes.

10. The apparatus of claim 1, wherein the multi-electrode pacemaker lead comprises a tapered nose cone on a first end, or wherein at least a portion of the length of the multi-electrode pacemaker lead is tapered toward the first end, with a diameter decreasing toward an end of the multi-electrode pacemaker lead that is configured to be inserted into the myocardium first during implantation.

11. The apparatus of claim 1, wherein the multi-electrode pacemaker lead comprises a detachable pull mechanism for implantation, and wherein the detachable pull mechanism is adapted to receive a retrograde tether configured to pull the multi-electrode pacemaker lead antegrade in during implantation of the multi-electrode pacemaker lead within the myocardium of the wall of the ventricle the heart.

12. The apparatus of claim 1, wherein the multi-electrode pacemaker lead includes a fracture site that is configured to be positioned at a myocardial-blood-space interface when implanted within the myocardium of the wall of the ventricle of the heart.

13. A method, comprising:
generating electrical impulses via a pulse generator;
transmitting the electrical impulses from the pulse generator to a plurality of ring electrodes of a pacing lead embedded within myocardium of a heart via a single channel or plurality of channels; and
applying the electrical impulses directly to the myocardium via the plurality of ring electrodes, wherein at least one of the plurality of ring electrodes is in circumferential contact with the myocardium.

14. The method of claim 13, wherein the pacing lead is embedded wholly or partially within the myocardium of a left ventricular wall or interventricular septum in proximity to a conduction system of the heart, and wherein applying the electrical impulses directly to the myocardium via the plurality of ring electrodes comprises applying the electrical impulses directly to the myocardium of the left ventricular wall of the heart.

15. The method of claim 13, wherein the pacing lead encircles at least 180 degrees around a base of a left ventricle of the heart, and wherein applying the electrical impulses directly to the myocardium via the plurality of ring electrodes comprises applying base-to-apex pacing throughout an entirety of the left ventricle.

16. The method of claim 13, wherein generating the electrical impulses via the pulse generator comprises tuning each of the electrical impulses based on a channel of the pulse generator, a channel output tuning stored in memory, and a desired type of pacing.

17. The method of claim 16, wherein the desired type of pacing is unipolar pacing or bipolar pacing.

18. The method of claim 13, wherein a portion of the pacing lead is embedded within the myocardium of an intraventricular septum of the heart, and the method further comprises applying the electrical impulses directly to a portion of a cardiac conduction system of the heart, and wherein the portion of the cardiac conduction system comprises a His bundle or wherein the portion of the cardiac conduction system comprises a left bundle branch conduction system.

19. A method for implanting a multi-electrode lead, comprising:
advancing a guidewire into myocardium via transvascular entry, the guidewire extending an inner lumen of the multi-electrode lead;
navigating the guidewire through the myocardium at a desired base-to-apex level of a left ventricle of the heart to define a desired trajectory for implanting the multi-electrode lead;
advancing the multi-electrode lead into the myocardium along the guidewire;
adjusting a rotational circumferential position of the multi-electrode lead along the desired trajectory until a desired rotational circumferential position of at least one electrode of the multi-electrode lead is achieved; and
withdrawing the guidewire and leaving the multi-electrode lead within the myocardium at the desired rotational circumferential position of the at least one electrode of the multi-electrode lead.

20. The method of claim 19, wherein navigating the guidewire through the myocardium at the desired base-to-apex level of the left ventricle of the heart comprises obtaining deep intracameral or intramyocardial guidewire purchase.
